(19) United States
(12) Patent Application Publication
(10) Pub. No.: US 2013/0319448 A1
(43) Pub. Date: Dec. 5, 2013

(54) HAIR CARE PRODUCT AND A METHOD OF USING SAID PRODUCT

(71) Applicant: Fredrik Olofsson, Hagersten (SE)

(72) Inventor: Fredrik Olofsson, Hagersten (SE)

(21) Appl. No.: 13/985,921

(22) PCT Filed: Feb. 14, 2012
(86) PCT No.: PCT/SE12/50145

§ 371 Date: Aug. 16, 2013

(30) Foreign Application Priority Data
Feb. 18, 2011 (SE) 1150134.3

(51) Int. Cl. A61K 8/64; A61Q 5/12

(52) U.S. Cl. CPC A61K 8/64; A61Q 5/12
USPC 132/202; 424/70.13; 424/70.14

(57) ABSTRACT

A hair care product to be used for protection and care of long hair when shampooing, and a method of using the same, wherein the product comprises the following constituents:—sorbitol, as a moisture preserving and a softening agent, in an amount of from 4 to 12% by weight; a thickener, such as guar flour, cellulose containing substances and/or gum arabicum, acting as an anti-static, film forming and surfactant agent, and as a conditioning agent, in an amount of from 1 to 5% by weight, silk protein and/or a degraded product thereof, as an anti-static, softening and antifoam agent in an amount of from 0.5 to 8% by weight, a film-forming, caring and re-moisturizing agent such as algae extract in an amount of from 1 to 2% by weight, a pH controlling and re-sealing agent, sealing agent, such as citric acid, vinegar, or similar, in an amount which gives the product a pH of about 4.4, and the remaining amount being water and optional additives, such as perfume, essential oils and similar.

### FIELD OF THE INVENTION

The present invention relates to a hair care product, especially a product intended for the protection and care of long hair when shampooing it, as well as a method of using said product.

### BACKGROUND

Many people dream of a long and healthy hair. The length of the growing phase and how long the hair becomes, are decided by our genes, and as a mean value a hair grows during seven years before it falls off. External influence such as styling with for example a blow-dryer or flat-irons can bring about heavy wear and the hair therefore need to be cut more often in order to look healthy and obtain a soft feeling. By using heat protecting products and lower heat this wear of the hair can be reduced to a certain extent, but what is mostly forgotten is that daily shampooing, despite all nourishing and re-moisturizing species in professional shampoos, also contributes to the drying-out, cleavage or even ripping off of the top. A hair having grown to a length of about 80 cm and having been washed every third day has been subjected to a shampoo at least 1 000 times at the top.

Already now there is a tendency that women look for an alternative to shampoo, when they experience that the cleaning wears the hair down, and instead they start to wash their hair with balm only (the old trick that like dissolves like, fat dissolves fat). At the beginning, one does not recognize the feeling of no lather, and this can be a less nice experience, since the skin on the scalp still overproduces fat, but gradually the production of tallow is restored, and balm is felt as a complete alternative to shampoo. The problem with this solution is that balm on the scalp in most cases does not give the same lightness and volume as a cleaning shampoo does.

There is also a dislike of choosing protein based hair care products, despite that the protein is what really strengthens the hair. Often the user does not notice an immediate improvement and rather chooses products with more fat making the hair glossy and soft, but, however, only cosmetically.

### THE INVENTION

One object of the present invention is to provide a product which solves or at least ameliorates the above noted problems in that it allows the user to continue to use a shampoo for cleaning while at the same time it protects the hair from the wear incurred by the cleaning.

This and other objects are achieved with the product according to the present invention with the characterizing features of claim 1. Further developments and preferred embodiments of the invention are defined in the sub claims.

The product according to the invention is a supplement to shampoo and balm and protects the lengths of the hair. It is a supplement to the daily hair care which spares the lengths from the wear of the shampoos. With this product in a hair care regime the user obtains it all, i.e. clean hair, filled with protein and gloss.

The invention also relates to a method of use of the product according to the invention in that it is applied in wet hair, on the lengths, before starting to shampoo, whereupon the hair is subsequently shampooed in the usual way, but only or mainly at the scalp. Thereafter both products are rinsed away and balm is applied as usual. The hair care product according to the invention, sometimes called primer, will protect the lengths from shampoo at rinsing, something that would not be the case when using only shampoo. The lengths will of course not be cleansed deeply in the same way, but the lengths do not have the same needs either, since fat is accumulated closest to the scalp. Simultaneously, however, the primer will still dissolve not too heavy dirt.

The product formula according to the invention will simply comprise the positive or nourishing properties of a shampoo for hair, but all reacting ingredients are excluded. The ingredients of the product counteract the washing effect and thereby also the lathering. A low pH value makes the hair to be sealed, which prevents the surface active agents of the shampoo from penetrating into the depth of the hair. The voids in the hair will be filled with protein. Moreover ingredients are added in order to obtain a higher surface tension in the water.

The product according to the invention will on the one hand quickly build up an impaired hair, on the other hand it prevents the hair from being damaged by washing. Simultaneously the hair closest to the scalp will receive the cleaning which is necessary to easily obtain the desired volume.

Further objects, advantages and characteristics of the invention will be evident from the following detailed description.

### DETAILED DESCRIPTION OF THE INVENTION

The invention is a hair care product intended to be used by people with long hair, especially in connexion with shampooing, but, as will be evident from the following description, it can also be used in other situations in order to protect the lengths of the hair.

One important property of the product according to the invention is that it has only protecting properties and accordingly does not have any cleansing effect. To the contrary, the product should render it difficult for active detergent substances to get into contact with the lengths of the hair.

The hair care product according to the invention is a product which is used before shampooing but possible also, as will be evident from below, for protecting the hair in connection with other activities, comprises
a substance with moisturizing and softening properties,
an anti-static binding agent, which is film-forming, regulates the viscosity, and is a surfactant,
a conditioning agent,
an anti-static, softening and antifoam agent
a film-forming, caring, and resealing agent,
a pH controlling agent,
the rest being water and possible additives, such as perfume, essential oils, etc.

One agent might fulfil several of the above functions.

The hair care product might contain optional other agents, such as perfumes, other fragrant substances, essential oils. Preservatives might occur and do not affect the action of the product.

More specifically, the hair care product according to the invention contains
sorbitol, as a moisture preserving and a softening agent, in an amount of from 4 to 12% by weight;
a thickener, such as guar flour, cellulose containing substances and/or gum arabicum, acting as an anti-static, film forming and surfactant agent, and as a conditioning agent, in an amount of from 1 to 5% by weight,
silk protein or a degraded product thereof as an anti-static, softening and antifoam agent in an amount of from 0.5 to 8% by weight,
a film-forming, caring and re-moisturizing agent such as algae extract in an amount of from 1 to 2% by weight,
a pH controlling and re-sealing agent, such as citric acid, vinegar, or similar, in an amount which gives the product a pH of about 4.4, and
remaining amount being water and optional additives, such as perfume, essential oils and similar.

According to a more specific embodiment of the invention, the hair care product according to the invention comprises:

| | |
|---|---|
| sorbitol | 5-9 |
| a thickener | 1-3 |
| silk protein or | 0.5-4 |
| a degraded product thereof | |
| a re-moisturizing/caring substance | 1-2 |
| a pH controlling agent such as | to pH about 4.4 |
| citric acid, vinegar or similar | |

the rest up to 100% is comprised of water and optional further additives, such as perfume, essential oils and similar, and optionally a preservative.

Guar flour in the form of hydroxy propyl guar can be used as anti-static, film forming, viscosity controlling and surfactant agent, in combination with hydroxypropyl guar-hydroxyl propyl trimonium chloride as conditioning and viscosity controlling agent. Alternative thickeners are for example cellulose based material, such as hydroxyetylcellulose, and gum arabicum.

The silk protein might be cocodimonium hydroxyl propyl hydrolyzed silk. Further, a degraded silk protein can be used, such as silk amino acids. Silk amino acids penetrates into the hair an add moisture, and also improves the plasticity of the hair, and make it less prone to breaking. Quaternary silk amino acids will attach more easy to the surface of the hair compared to not quaternized silk amino acids, they are even more conditioning, and they are effective at very low concentrations.

The algae extract might be hydrolyzed rodophycea extract, possibly in combination with chondrus crispus extract, and/or oligogeline (carrageenan) might be used. A further caring agent is Limnanthes Alba Seed oil.

The product according to the invention will obstruct the cleaning effect of surfactants. It accomplishes that the hair is sealed (the scale layer is sealed) because of a low pH value, while at the same time is gives gloss, moisturizes, and adds protein.

The product should only be used in the lengths out to the top, starting at a distance from the scalp, and it will attach to the hair lengths as a protective cover/film/cream, before washing the hair. The product shall remain during shampooing and is intended to be used in connection with hair washing

The product is suitable for long natural hair, chemically treated hair, and also for people with false hair/hair extensions and also people having scalp problems. Furthermore, the product will act excellently as an extra protection under a bathing cap in order to prevent chlorine water from entering into the hair.

The product spares effectively the hair lengths when using dandruff shampoo, which is very deep-cleansing and drying-out.

People being subject to lice in the scalp often wash the hair so that the individual hairs are destroyed, since agents against lice are very strong. The product gives a good protection against this effect too in the lengths.

The invention is further illustrated in the following with specific examples of specific compositions. It should however be noted that they are given only as illustrative examples, not limiting the scope to the invention. The contents of the different ingredients are expressed in % by weight.

### EXEMPLE 1

| | |
|---|---|
| Mackernium 261 (Guar Hydroxy propyl trimonium Chloride)¹ | 1.9 |
| C*Sorbidex NC 16207 (Sorbitol)² | 7 |
| Crosilkquat (Cocodimonium Hydroxypropyl Silk Amino Acids)³ | 3.1 |
| Oligogeline (Carrageenan)² | 1.8 |
| Citric Acid | 0.05 |
| Euxyl PE 9010 (Phenoxyethanol, Ethylhexylglycerine)⁴ | 0.4 |
| Perfume Longanfruit 44756 (Perfume)⁵ | 0.04 |

Remaining part up to 100% is water and unavoidable impurities.

### EXEMPLE 2

| | |
|---|---|
| Mackernium 261 (Guar Hydroxypropyltrimonium Chloride)¹ | 1.9 |
| C*Sorbidex NC 16207 (Sorbitol)² | 5 |
| Crosilkquat (Cocodimonium hydroxypropyl Silk Amino Acids)³ | 3.1 |
| Oligogeline (Carrageenan)² | 1.8 |
| Citric Acid | 0.05 |
| Euxyl PE 9010 (Phenoxyethanol, Ethylhexylglycerine)⁴ | 0.4 |
| Perfume Longanfruit 44756 (Perfume)⁵ | 0.04 |
| Remaining part up to 100% is water and unavoidable impurities. | |

Suppliers:
¹Vendico;
²Brenntag;
³Croda;
⁴Bionord;
⁵Kavat Kosmetik.

### EXAMPLE 3

| | |
|---|---|
| Sorbitol | 7.19 |
| Crosilkquat LQ | 3.04 |
| Cosmedia Guar C 261 N | 1.49 |
| Natrosol 250 HHR | 0.35 |
| Oligogeline PF | 0.300 |
| Meadofoam Seed Oil | 2.45 |
| Citric Acid mono fine | 0.23 |
| Euxyl K-712 | 1.000 |
| Perfum Intensive Care | 0.18 |
| Water | up to 100% |

### THE METHOD ACCORDING TO THE INVENTION

There are several different ways of washing hair:

Normal wash with shampoo and balm:
Wet the hair
Add tensides/sulphates and massage up a lather/foam
Rinse
Add balm
Rinse

In this case, all natural fats in the hair are washed away, said natural fats being the best protection for the hair, and adds artificial substances for the hair not to get entangled.

The balm method:
Wet the hair
add balm and massage the scalp and the hair
Rinse

A person having very dry hair can choose to wash the hair only with balm, according to the thesis like dissolves like, fat dissolves fat. On a thick, dry, naturally curly hair, this might well be a good solution, but for those having flat hair, the lift/feeling of clean hair will not be sufficient at the scalp, and will therefore fall flat.

### METHOD ACCORDING TO THE INVENTION

Rinse/soak the hair (here the water will dissolve light dirt, dust and particles, and water soluble hair styling products),
Apply the hair care product according to the invention (“the primer”) on the length of a distance from the scalp all the way to the top,
Wash the scalp while avoiding to draw the shampoo out onto the primer.
Rinse all of the hair, whereby the water and shampoo will bring the primer with them. Now the scalp is clean and the primer having been sitting in the hair for a while has had time to neutralize odours, add proteins and seal the individual hairs.
Finish with balm for extra moisture and disentanglement.

Accordingly, in this case only the scalp is washed, the lengths are protected from tensides and sulphates, the proteins will enter into the lengths of the hair which strengthens the hair, the scale layer is sealed, and the procedure is finished with balm/disentanglement and extra gloss.

With the method according to the invention, the top/length reaching out longer than about 10 cm is not washed. The human hair grows about 8-10 cm in a year. This is about as long the hair can take daily shampooing without becoming split, cracked and get a dry feeling. Hair, which is older that about 1 year, will accordingly not be washed. The intention is that the length will rest from tensides as long as it is growing.

The method according to the invention is suitable also for those who uses extensions of the hair, since natural fat, which is the best lubricant for the hair, seldom can reach past the knots, where the hair is fastened. These lengths will therefore not obtain a natural lubrication, and will therefore dry out extremely fast. With the product according to the invention the lifetime can be extended also for false hair.

The product according to the invention is thus only applied onto the lengths and not on the skin of the scalp, and it protects and takes care of the hair.

The same washing effect will be experienced in the scalp and the hair closest to the scalp, but the length will be completely protected from washing.

It should be specifically pointed out that contrary to known products, the hair care product according to the invention shall not be rinsed away before shampooing.

If someone is staying in a dirtier environment than usual, and therefore feels a desire to wash also the lengths of hair, the shampoo is drawn out onto the product according to the invention sitting on the lengths of the hair, and the hair will then receive a gentler cleaning than if the product was not used. The low pH value makes it more difficult for the tensides to work, when they reach the protective layer on the separate hairs, with which is accomplished that the cleaning will be milder than if only shampoo is used.

An added value is that the shampoo consumption will be reduced, and it will be at least halved, since now only the scalp is to be washed, while at the same time the environmental load will be reduced, since the product does not contain tensides, and thanks to reduced use of shampoo the amount of tensides let out will be correspondingly reduced.

There is also the thought that optionally prevalent fragrances are only intended to neutralize non-wanted odours in the hair, such as smoke, perspiration, or alternatively in order to match the shampoo and balm, with which the product is launched.

1. A hair care product to be used for protection and care of long hair when shampooing, characterized in that it comprises the following constituents:
sorbitol, as a moisture preserving and a softening agent, in an amount of from 4 to 12% by weight;
a thickener, such as guar flour, cellulose containing substances and/or gum arabicum, acting as an anti-static, film forming and surfactant agent, and as a conditioning agent, in an amount of from 1 to 5% by weight,
silk protein and/or a degraded product therefrom, as an anti-static, softening and antifoam agent in an amount of from 0.5 to 8% by weight,
a film-forming, caring and re-moisturizing agent such as algae extract in an amount of from 1 to 2% by weight,
a pH controlling and re-sealing agent, such as citric acid, vinegar, or similar, in an amount which gives the product a pH of about 4.4, and
the remaining amount being water and optional additives, such as perfume, essential oils and similar.

2. The hair care product according to claim 1, wherein the product is comprised of, in % by weight:

| | |
|---|---|
| sorbitol | 5-9 |
| a thickener | 1-3 |
| silk protein, and/or a degraded product thereof | 0.5-4 |
| a re-moisturizing/caring substance | 1-2 |
| a pH controlling agent such as citric acid, | to pH about 4.4 |
| vinegar or similar | |

the rest up to 100% is comprised of water and optional further additives, such as perfume, essential oils and similar, and optionally a preservative.

3. A method for the protection and care of long hair when shampooing it with a product according to claim 1, characterized in that it comprises the following steps of:
i) rinsing/soaking the hair,
ii) applying the product according to claim 1 on the lengths about 10 cm from the scalp and all the way out towards the top,
iii) washing the scalp while avoiding drawing shampoo out over the hair lengths treated in step i),
iv) rinsing all of the hair, whereby the water and shampoo will bring with them the product sitting on the hair lengths, resulting in that the scalp is clean and the product being present during the shampooing having had time to neutralize odors, add proteins and seal each hair, and
v) optionally, finishing with balm for extra moisture and disentanglement.
